(19) United States
(12) Patent Application Publication
(10) Pub. No.: US 2004/0006244 A1
(43) Pub. Date: Jan. 8, 2004

(54) Process for making ethyencially unsaturated acids and acids

(76) Inventor: Leo Ernest Manzer, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/437,759

(22) Filed: May 14, 2003

Related U.S. Application Data
(60) Provisional application No. 60/382,526, filed on May 22, 2002.

(51) Int. Cl. C07C 57/02; C07C 69/52

(52) U.S. Cl. 560/205; 562/598

(57) ABSTRACT

The present invention relates to production of ethylenically unsaturated acids and ethylenically unsaturated esters by the catalytic reaction of an alkanoic acid or an alkanoic ester, with formaldehyde, in presence of a basic catalyst and optionally in the presence of an alcohol. More specifically, this invention relates to production of methyl methacrylate, an ester, by reaction of methyl propionate with formaldehyde, in presence of a basic catalyst, for example, cesium acetate on silica. This invention discloses a process for obtaining a high conversion of ethylenically unsaturated acids and esters.

### FIELD OF INVENTION

The present invention relates to production of ethylenically unsaturated acids or esters by the catalytic reaction of an alkanoic acid or ester, with formaldehyde, in presence of a basic catalyst, and optionally in the presence of an alcohol. More specifically, this invention relates to production of methyl methacrylate, an ester, by reaction of methyl propionate with formaldehyde, in presence of a basic catalyst, for example, cesium acetate on silica. This invention discloses a process for obtaining a high conversion of ethylenically unsaturated acids and ethylenically unsaturated esters.

### BACKGROUND OF THE INVENTION

Methyl methacrylate is the precursor of poly methyl methacrylate. Poly methyl methacrylate is used in wide variety of plastic applications ranging from windows to biocompatible materials. Efficient preparation of a,b-unsaturated aliphatic monocarboxylic acid or an ester from a saturated aliphatic monocarboxylic acid or an ester and formaldehyde is required to make the process commercially attractive. Brydson, J. A., “Plastics Materials,” 5^{th} Edition, Butterworth Heinemann Ltd., Oxford, UK, lists uses and properties of poly methyl methacrylate and other acrylic based materials, hereby incorporated by reference.

Methyl methacrylate is an ethylenically unsaturated acid. Production of ethylenically unsaturated acids or esters by the catalytic reaction of an alkanoic acid or ester with formaldehyde is known in the art. For example, patent publication WO 99/052628 discloses a process for manufacturing methyl methacrylate from methyl propionate, formaldehyde and methanol reactants in the presence of a catalyst comprising silica and 1% to 10% by weight of cesium. Preferred catalyst content for this process is about 2%. The catalyst contains zirconium dispersed in the porous silica. Although a high product yield is desired, the maximum yield obtained by this process is about 12% to 13%. Through stoichiometric conversion, this translates to a maximum theoretical conversion of methyl propionate based on formaldehyde of 64%.

EP 0,265,964 discloses a process for preparing an a,b-unsaturated aliphatic monocarboxylic acid or an ester from a saturated aliphatic monocarboxylic acid or an ester and formaldehyde. Silica supported catalysts containing antimony and alkali metals are used in the reaction. The maximum percent yield of propionic acid based on formaldehyde is about 40%.

U.S. Pat. No. 3,933,888 discloses the production of methyl methacrylate by the reaction of EP 0,265,964 using a catalyst formed by calcining a pyrogenic silica with a base such as a cesium compound, and indicates that the pyrogenic silica may be mixed with 1-10% by weight of pyrogenic zirconia. That reference also discloses the use of a catalyst made from a composition containing cesium as the alkali metal and a small amount of borax. In “Applied Catalysis”, 102, (1993) p. 215-232, Yoo discloses catalysts of cesium supported on silica doped with various modifiers. U.S. Pat. No. 3,933,888 indicates the importance of using pyrogenic silica as a catalyst support in the process of EP 0,265,964 and demonstrates that other types of silicas are unsuitable.

The present invention discloses a process for making ethylenically unsaturated acids and ethylenically unsaturated esters by the catalytic reaction of an alkanoic acid or ester from methyl propionate and formaldehyde reactants. The present invention discloses a process for obtaining such acids and esters, specifically methyl methacrylate, with a surprising result providing significantly higher conversion efficiency afforded by hitherto known processes used for producing methyl methacrylate from methyl propionate and formaldehyde. The present invention uses a catalyst range higher than what has been accepted as normal in currently accepted standard processes.

The process of the invention enables the conversion of about 60% to about 70% of methyl propionate. This conversion rate is twice that of currently reported processes. Therefore, this invention provides a novel, commercially efficient process for the manufacture of methyl methacrylate.

### SUMMARY OF THE INVENTION

This invention relates to a process for preparing ethylenically unsaturated acids, the process comprising the step of contacting an alkanoic acid of the formula R′—CH₂—COOR, with formaldehyde in the presence of a heterogeneous base catalyst; wherein R and R′ are each, independently hydrogen or an alkyl group with 1 to 4 carbon atoms, and wherein the catalyst concentration is from about 12% to about 20% of the original weight of the reactants.

This invention also relates to a process for preparing ethylenically unsaturated esters, the process comprising the step of contacting an ester of an alkanoic acid, of the formula R′—CH₂—COOR, with formaldehyde in the presence of a heterogeneous base catalyst; wherein R and R′ are each, independently hydrogen or an alkyl group with 1 to 4 carbon atoms, and wherein the catalyst concentration is from about 12% to about 20%.

Further, this invention relates to a process for preparing methyl methacrylate or methacrylic acid from a reaction between methyl propionate or propionic acid, respectively, and formaldehyde in presence of basic heterogeneous catalyst in a concentration range of from about 12% to about 20%. Methanol, or ethanol may be optionally present as media for this reaction.

### DETAILED DESCRIPTION OF THE INVENTION

This invention relates to the synthesis of ethylenically unsaturated acids or esters thereof, in the presence of catalysts. More specifically, this invention relates to synthesis of methacrylic acid or alkyl methacrylates, from alkyl propionate and formic acid. The ethylenically unsaturated acids of the invention are made by reacting an alkanoic acid of the formula R′—CH₂—COOR, where R′ and R are each, independently, hydrogen or an alkyl group, especially a lower alkyl group containing for example 1-4 carbon atoms, with formaldehyde. The ethylenically unsaturated esters of the invention are made by reacting an alkanoic ester of the formula R′—CH₂—COOR, where R′ and R are each, independently, hydrogen or an alkyl group, especially a lower alkyl group containing for example 1-4 carbon atoms, with formaldehyde.

Methacrylic acid is made by the catalytic reaction of propionic acid, with formaldehyde in accordance with the reaction scheme shown below. Alkyl ester is made by the catalytic reaction of the corresponding alkyl ester with formaldehyde. Therefore, methacrylic acid is made by the catalytic reaction of propionic acid with formaldehyde in accordance with the following reaction scheme:

General Reaction:

CH₃—CH₂—COOR+HCHO→CH₃—CH(CH₂OH)—COOR

CH₃—CH(CH₂OH)—COOR→CH₃—C(:CH₂)—COOR+H₂O

Also, methyl methacrylate, is made by the catalytic reaction of methyl propionate, with formaldehyde in accordance with the following reaction scheme:

Reaction for Methyl Methacrylate Synthesis:

CH₃—CH₂—COOCH₃+HCHO→CH₃—CH(CH₂OH)—COOCH₃

CH₃—CH(CH₂OH)—COOCH₃→CH₃—C(:CH₂)—COOCH₃+H₂O

The catalyst useful in the process of the invention is a substance that affects the rate of the reaction but not the reaction equilibrium, and emerges from the process chemically unchanged. A chemical promoter generally augments the activity of a catalyst. As used herein, by “promoter” is meant a compound that is added to enhance the physical or chemical function of a catalyst. By “metal promoter” is meant a metallic compound that is added to enhance the physical or chemical function of a catalyst. The promoter herein may be incorporated into the catalyst during any step in the chemical processing of the catalyst constituent. The chemical promoter generally enhances the physical or chemical function of the catalyst agent, but can also be added to retard undesirable side reactions.

A suitable base catalyst can be defined either as a substance which has the ability to accept protons as defined by Bronsted, or as a substance which has an unshared electron pair with which it can form a covalent bond with an atom, molecule or ion as defined by Lewis. A further definition of base catalysts and how to determine if a particular substance is base is explained in Tanabe, K., Catalysis: Science and Technology, Vol. 2, pg. 232-273, ed. Anderson, J. and Boudart, M., Springer-Verlag, N.Y., 1981. Examples of suitable base catalysts include, but are not limited to, metal oxides, hydroxides, carbonates, silicates, phosphates, aluminates and mixtures thereof. Preferred are metal oxides, carbonates, and silicates. More preferred are Group 1 and Group 2 metals of the Periodic Table of Elements, and rare earth oxides, carbonates, and silicates. The catalysts of the invention can be obtained commercially or be prepared from suitable starting materials using methods known in the art.

The catalysts employed herein may be used as powders, granules, beads or other particulate forms, or may be supported on an essentially inert support as is common in the art of catalysis. Selection of an optimal average particle size for the catalyst will depend upon such process parameters as reactor residence time and desired reactor flow rates. The catalyst is preferably used in the form of a fixed bed so it is therefore desirable that the composition of the catalyst is formed into shaped units. Shapes such as spheres, granules, pellets, aggregates, or extrudates, typically having maximum and minimum dimensions in the range 1 to 10 mm are useful herein, however the particular shape of the catalyst is not critical to the invention. The composition may be so shaped at any stage in the production of the catalyst.

The metal catalyst used in the process disclosed may be used as a supported or as an unsupported catalyst. A supported catalyst is one in which the active catalyst agent is deposited on a support material by spraying, soaking or physical mixing, followed by drying, calcination, and if necessary, activation through methods such as reduction or oxidation. Materials frequently used as catalyst supports are porous solids with high total surface areas (external and internal) which can provide high concentrations of active sites per unit weight of catalyst. The catalyst support may enhance the function of the catalyst agent. As used herein, an “unsupported catalyst” is a catalyst that is not supported on a catalyst support material.

Any method known in the art to prepare supported catalysts can be used to prepare the supported catalysts of the invention. The catalyst support material can be neutral, acidic or basic, as long as the surface of the catalyst/catalyst support combination is basic. Preferred catalyst supports are those which are neutral and have low surface areas. Commonly used techniques for treatment of catalyst supports with metal catalysts can be found in B. C. Gates, Heterogeneous Catalysis, Vol. 2, pp. 1-29, Ed. B. L. Shapiro, Texas A & M University Press, College Station, TX, 1984, hereby incorporated by reference.

The catalysts of the present invention may further include additives and 10 promoters, which will enhance the efficiency of the catalyst. Use of these materials is common and well known in the art (see for example, Kirk-Othmer Encyclopedia of Chemical Technology, Howe-Grant Ed., Vol. 5, pg. 326-346, (1993), John Wiley & Sons, New York and Ullmann's Encyclopedia of Industrial Chemistry, Vol. A5, Gerhartz et al., Eds., pp. 337-346, (1986), VCH Publishers, New York). The relative percentage of the catalyst promoter in the reaction of the invention can vary from about 0.01% to about 50.00% by weight of catalyst

A preferred group of catalysts useful in the process of the invention are metal silicates. By “silicate” is meant an anion consisting of Si, O, and optionally H. These include but are not limited to SiO₃⁻², Si₂O₇⁻⁶, and SiO₄⁻⁴, and their various hydrated forms. More preferred are silicate salts of Group 2 metals; most preferred is magnesium silicate. One particularly preferred catalyst is Magnesol® magnesium silicate, a hydrated, synthetic, amorphous form of magnesium silicate produced by The Dallas Group of America, Inc.

Other preferred groups of catalysts include oxides, carbonates, and mixture thereof, of Group 1 and Group 2 metals of the Period Table, and rare earth metal, optionally supported on a suitable support. One method to prepare these catalysts involves dissolving a metal acetate salt in water. A catalyst support, such as silica, is soaked in the solution, then calcined. The metal acetate salt is thereby oxidized to an oxide, carbonate, or a mixture thereof.

Metals from Group 1 and Group 2 of the Periodic Table are also alternative preferred catalysts useful in the process of the invention. Barium, cesium, or rubidium are most preferred. Potassium is also a preferred alkali metal.

Preferred catalyst support materials of the invention are selected from the group consisting of carbon, alumina, titania, silica, zirconia, zeolites, clays, silica-alumina, Ka-160, calcium carbonate, and combinations thereof. A preferred catalyst support is silica. Gel silicas are preferred although suitable pyrogenic silicas may also be used. The catalysts may be made by impregnating silica particles of the physical dimensions required of the catalyst with a solution of suitable compounds, e.g. salts, of the modifier element in a suitable solvent, followed by drying. The impregnation and drying procedure may be repeated more than once in order to achieve the desired additive loading. Loading is defined as the weight of catalyst measured as a percentage of the total weight of the catalyst and catalyst support.

The catalyst concentration of the invention is preferably from 12% to 20% by weight of the alkali metal in the reactant. Catalyst concentration of from 14% to about 18% is further preferred.

According to the process of the invention, the alkanoic acid or ester thereof and formaldehyde are fed either directly or after mixing to the reactor containing the catalyst. The molar ratio of alkanoic acid or ester to formaldehyde is preferably from about 0.1/1 to 10/1 at the beginning of the reaction. A molar ratio of about 0.1/4 at the beginning of the reaction is further preferred

The formaldehyde of the invention is added to the reaction mixture in any suitable form, including but not limited to aqueous formaldehyde solutions, or anhydrous formaldehyde derived from a formaldehyde drying procedure, trioxane, diether of methylene glycol and paraformaldehyde. For ease of administration, the formaldehyde herein can also be introduced in the form as formalin because formalin is commercially available and inexpensive.

Optionally, water may be added to the reaction mixture during the process of the invention. The water content can range up to about 50% by weight of the reaction mixture.

When the desired product is an unsaturated ester made by reacting an ester of an alkanoic acid ester with formaldehyde, an alcohol corresponding to the ester may also be fed to the reaction mixture. The alcohol may be fed either independently or together with the other components. The alcohol serves to reduce the quantity of acids leaving the reactor. In order to effect the conversion of acids to their respective esters without depressing catalyst activity, alcohol can be added at any time during the reaction. The formaldehyde in the process of the invention can also be added as a mixture of formaldehyde in an alcohol. The concentration of formaldehyde in the alcohol ranges from about 20% to about 60%. Preferably, the alcohol is methanol or ethanol. Formaldehyde content of from about 20% to about 50% is preferred in methanol. Formaldehyde content of from about 25% to about 55% is preferred in ethanol.

In a preferred embodiment of the invention, methyl propionate is utilized as the reactant with a 4:1 ratio of (25% formaldehyde in ethanol) to methyl propionate, and about 15% cesium oxyacetate as catalyst supported on silica.

Therefore, in a preferred embodiment of the reaction of the invention, methyl methacrylate is produced by feeding methyl propionate, methanol or ethanol, formaldehyde and water to the catalyst. Suitable catalysts include alkali metal-doped, especially cesium-doped, silica catalysts in the range of 14% to 16% by weight. The catalysts are then preferably calcined before use, for example in air, at a temperature in the range 300° C. to 600° C., particularly at 500° C. to 550° C.

The process of the invention is preferably performed in the gas phase. The process can be performed in any suitable reactor such as a pulse, fluidized bed, fixed bed, steady state riser reactor, and a re-circulating solids reactor system. The reaction temperature is preferably about 250° C. to about 500° C., more preferably about 300° C. to about 400° C., most preferably from about 330° C. to about 390° C.°. The process is preferably performed at pressures of ambient to about 52 MPa. The residence time of the reaction mixture within the reactor is preferably from about 0.05 seconds to 180 seconds.

It will be appreciated that the selectivities and yields of product may be enhanced by additional contact with the catalyst. For example, yields and selectivities may increase where the reactor effluent containing a mixture of reactant and product may be passed one or more times over the catalyst under the reaction conditions to enhance the conversion of reactant to product.

The process of the instant invention may additionally comprise the recovery or isolation of ethylenically unsaturated esters. This can be done by any method known in the art, such as distillation, decantation, or filtration.

### EXAMPLES

The process of the invention is further illustrated by the following examples. Specifically, the following procedure illustrates preparation of base catalysts on silica supports. All metals herein were used in the acetate salt form.

Materials and Methods

The following abbreviations are used herein:

| | |
|---|---|
| F | Formalin-38% formaldehyde in water |
| MP | Methyl propionate |
| EP | Ethyl propionate |
| MeOH | Methanol |
| EtOH | Ethanol |
| CH2O | Formaldehyde |
| MMA | Methyl methacrylate |
| EMA | Ethyl methacrylate |
| CsOAc | Cesium Oxyacetate |
| CT | Contact time |
| N₂ | Nitrogen (cc/m) |
| STP | Standard temperature and pressure |
| Temp | temperature |
| TOS | time on stream |
| Conv. | Conversion |
| hr | hour(s) |
| m | meter(s) |
| mm | millimeter(s) |
| cc | cubic centimeter(s) |

### EXAMPLES 1-11

Experiment Procedure For Preparation Of 20% Cesium On Silica

Cesium acetate (2.91 g, Aldrich, Milwaukee, Wis.) was dissolved in H₂O (14 ml) and the solution was added dropwise into silica (8.07 g, W.R. Grace, Columbia, MD, Grade 55,12×20 mesh). The mixture was allowed to stand at room temperature for 2 hr and then the mixture was transferred into an alumina boat. The boat was placed in a horizontal quartz tube and purged with air. The supported catalyst was heated at 120° C. for 4 hr and then at 450° C. for 16 hr in a stream of air. The sample was then cooled to yield 9.87 g of 20% Cs on silica.

Methyl Methacrylate From Methyl Propionate and Formaldehyde Gas Phase Reaction

A premixed solution of methylpropionate and formaldehyde solution was added to a positive displacement pump and the liquid was fed directly into a 3/8″ (9.5 mm) ID stainless steel reactor. Nitrogen or air was metered into the reactor using mass flow controllers. The effluent from the reactor was collected in small sample vials, containing methanol or ethanol, that were cooled to 20° C. The analysis was then carried out on a Hewlett-Packard 5890 Gas Chromatograph (GC) using an RTX-1701 GC column, 30 m long and 0.53 mm ID. Conversions and selectivities are based on normalized areas.

The feed solution was a 2:1 molar ratio of formalin to methyl propionate for examples 1-6, and 4:1 for examples 7-11.

TABLE 1

| | | | | | | | | | | % |
|---|---|---|---|---|---|---|---|---|---|---|
| | | | | Feed | | | | | % | Theoretical |
| | | | | Molar | | | | | Conv. | Conv. |
| Ex. | | Feed | Feed | Ratio | N₂ | CT (s) | Temp | TOS | MP to | Based on |
| No. | Catalyst | Solution | cc/hr | F/MP | cc/m | STP | (° C.) | (hr) | MMA | CH₂O |
| 1 | 6758-22-03 15% | 0.2:1 | 2 | 0.2:1 | 48 | 1.5 | 340 | 30.08 | 2.07 | 10.33 |
| | Ba on Ka-160 | Formalin/MP | | | | | | | | |
| | calcined 550 c/air. | | | | | | | | | |
| 2 | 6758-22-03 15% | 0.2:1 | 2 | 0.2:1 | 48 | 1.5 | 340 | 0.25 | 2.27 | 11.36 |
| | Ba on Ka-160 | Formalin/MP | | | | | | | | |
| | calcined 550 c/air. | | | | | | | | | |
| 3 | E103003-034 | 0.2:1 | 2 | 0.2:1 | 48 | 6.15 | 340 | 0.5 | 15.35 | 76.77 |
| | 15% CsOAC on | Formalin/MP | | | | | | | | |
| | SiO2(grace | | | | | | | | | |
| | 55, 12 × 20mesh) | | | | | | | | | |
| 4 | E103003-034 | 0.2:1 | 1 | 0.2:1 | 24 | 12.3 | 340 | 0.3 | 12.59 | 62.95 |
| | 15% CsOAC on | Formalin/MP | | | | | | | | |
| | SiO2(grace | | | | | | | | | |
| | 55, 12 × 20mesh) | | | | | | | | | |
| 5 | E103003-034 | 0.2:1 | 1 | 0.2:1 | 24 | 12.3 | 340 | 0.5 | 12.17 | 60.84 |
| | 15% CsOAC on | Formalin/MP | | | | | | | | |
| | SiO2(grace | | | | | | | | | |
| | 55, 12 × 20mesh) | | | | | | | | | |
| 6 | E103003-034 | 0.2:1 | 1 | 0.2:1 | 24 | 12.3 | 340 | 1 | 11.84 | 59.21 |
| | 15% CsOAC on | Formalin/MP | | | | | | | | |
| | SiO2(grace | | | | | | | | | |
| | 55, 12 × 20mesh) | | | | | | | | | |
| 7 | E103003-034 | 0.4:1 | 2 | 0.4:1 | 48 | 6.15 | 340 | 0.75 | 22.67 | 56.68 |
| | 15% CsOAC on | Formalin/MP | | | | | | | | |
| | SiO2(grace | | | | | | | | | |
| | 55, 12 × 20mesh) | | | | | | | | | |
| 8 | E103003-034 | 0.4:1 | 2 | 0.4:1 | 48 | 6.15 | 340 | 1 | 22.78 | 56.95 |
| | 15%CsOAC on | Formalin/MP | | | | | | | | |
| | SiO2(grace | | | | | | | | | |
| | 55, 12 × 20mesh) | | | | | | | | | |
| 9 | E103003-034 | 0.4:1 | 2 | 0.4:1 | 48 | 6.15 | 340 | 2 | 22.49 | 56.22 |
| | 15% CsOAC on | Formalin/MP | | | | | | | | |
| | SiO2(grace | | | | | | | | | |
| | 55, 12 × 20mesh) | | | | | | | | | |
| 10 | E103003-034 | 0.4:1 | 2 | 0.4:1 | 48 | 6.15 | 380 | 0.75 | 14.48 | 36.21 |
| | 15% CsOAC on | Formalin/MP | | | | | | | | |
| | SiO2(grace | | | | | | | | | |
| | 55, 12 × 20mesh) | | | | | | | | | |
| 11 | E103003-034 | 0.4:1 | 2 | 0.4:1 | 48 | 6.15 | 380 | 0.75 | 14.48 | 36.21 |
| | 15% CsOAC on | Formalin/MP | | | | | | | | |
| | SiO2(grace | | | | | | | | | |
| | 55, 12 × 20mesh) | | | | | | | | | |

### EXAMPLES 12-22

Methyl Methacrylate From Methyl Propionate and Formaldehyde Gas Phase Reaction

A premixed solution of methylpropionate and formaldehyde solution was added to a positive displacement pump and the liquid was fed directly into a ⅜″ (9.5 mm) ID stainless steel reactor. Nitrogen or air was metered into the reactor using mass flow controllers. The effluent from the reactor was collected in small sample vials, containing methanol or ethanol, that were cooled to 20° C. The analysis was then carried out on a Hewlett-Packard 5890 Gas Chromatograph (GC) using an RTX-1701 GC column, 30 m long and 0.53 mm ID. Conversions and selectivities are based on normalized areas.

The feed solution was a 4:1 molar ratio of formaldehyde to methyl propionate for examples 12-17. The formaldehyde itself was a 50% solution in methanol. The feed solution was a 2:1 molar ratio of formalin to methyl propionate for examples 18-22.

The feed solution was a 4:1 molar ratio of formaldehyde to methyl propionate for examples 12-17. The formaldehyde itself was a 50% solution in methanol. The feed solution was a 2:1 molar ratio of formalin to methyl propionate for examples 18-22.

In example 17, 24 cc/min of air was used instead of nitrogen.

TABLE 2

| | | | | | | | | | | % |
|---|---|---|---|---|---|---|---|---|---|---|
| | | | | Feed | | | | | % | Theortetical |
| | | | | Molar | | | | | Conv. | Conv. |
| Ex. | | Feed | Feed | Ratio | N₂ | CT (s) | Temp | TOS | MP to | Based on |
| No. | Catalyst | Solution | cc/hr | F/MP | cc/m | STP | (° C.) | (hr) | MMA | CH₂O |
| 12 | 6758-22-03 | 4:1 50% | 1 | 4 1 | 24 | 3.1 | 300 | 0.67 | 6.44 | 6.44 |
| | 15% Ba on Ka- | CH2O in | | | | | | | | |
| | 160 calcined | MeOH/MP | | | | | | | | |
| | 550 c/air. | | | | | | | | | |
| 13 | 6758-22-03 | 4:1 50% | 1 | 4 1 | 24 | 3.1 | 340 | 0.17 | 16.76 | 16.76 |
| | 15% Ba on Ka- | CH2O in | | | | | | | | |
| | 160 calcined | MeOH/MP | | | | | | | | |
| | 550 c/air. | | | | | | | | | |
| 14 | 6758-22-03 | 4:1 50% | 1 | 4 1 | 24 | 3.1 | 380 | 0.33 | 1.65 | 1.65 |
| | 15% Ba on Ka- | CH2O in | | | | | | | | |
| | 160 calcined | MeOH/MP | | | | | | | | |
| | 550 c/air. | | | | | | | | | |
| 15 | 6758-22-03 | 4:1 50% | 2 | 4 1 | 48 | 1.5 | 340 | 0.08 | 2.86 | 2.86 |
| | 15% Ba on Ka- | CH2O in | | | | | | | | |
| | 160 calcined | MeOH/MP | | | | | | | | |
| | 550 c/air. | | | | | | | | | |
| 16 | 6758-22-03 | 4:1 50% | 2 | 4 1 | 48 | 1.5 | 340 | 0.5 | 4.47 | 4.47 |
| | 15% Ba on Ka- | CH2O in | | | | | | | | |
| | 160 calcined | MeOH/MP | | | | | | | | |
| | 550 c/air. | | | | | | | | | |
| 17 | 6758-22-03 | 4:1 50% | 1 | 4 1 | 0* | 3.1 | 340 | 0.33 | 16.76 | 16.76 |
| | 15% Ba on Ka- | CH2O in | | | 24 ccm | | | | | |
| | 160 calcined | MeOH/MP | | | air | | | | | |
| | 550 c/air. | | | | | | | | | |
| 18 | 6758-22-03 | 0.2:1 | 1 | 2 1 | 0 | 3.1 | 380 | 0.8 | 5.95 | 29.77 |
| | 15% Ba on Ka- | Formalin/MP | | | | | | | | |
| | 160 calcined | | | | | | | | | |
| | 550 c/air. | | | | | | | | | |
| 19 | 6758-22-03 | 0.2:1 | 1 | 2 1 | 0 | 3.1 | 350 | 0.08 | 3.48 | 17.38 |
| | 15% Ba on Ka- | Formalin/MP | | | | | | | | |
| | 160 calcined | | | | | | | | | |
| | 550 c/air. | | | | | | | | | |
| 20 | 6758-22-03 | 0.2:1 | 1 | 2 1 | 0 | 3.1 | 350 | 0.25 | 3.87 | 19.34 |
| | 15% Ba on Ka- | Formalin/MP | | | | | | | | |
| | 160 calcined | | | | | | | | | |
| | 550 c/air. | | | | | | | | | |
| 21 | 6758-22-03 | 0.2:1 | 1 | 2 1 | 0 | 3.1 | 350 | 2 | 2.95 | 14.75 |
| | 15% Ba on Ka- | Formalin/MP | | | | | | | | |
| | 160 calcined | | | | | | | | | |
| | 550 c/air. | | | | | | | | | |
| 22 | 6758-22-03 | 0.2:1 | 1 | 2 1 | 0 | 3.1 | 380 | 0.25 | 2.13 | 10.64 |
| | 15% Ba on Ka- | Formalin/MP | | | | | | | | |
| | 160 calcined | | | | | | | | | |
| | 550 c/air. | | | | | | | | | |

### EXAMPLES 23

Methyl Methacrylate From Methyl Propionate and Formaldehyde Gas Phase Reaction

A premixed solution of methylpropionate and formaldehyde solution was added to a positive displacement pump and the liquid was fed directly into a 3/8″ (9.5 mm) ID stainless steel reactor. Nitrogen or air was metered into the reactor using mass flow controllers. The effluent from the reactor was collected in small sample vials, containing methanol or ethanol, that were cooled to 20° C. The analysis was then carried out on a Hewlett-Packard 5890 Gas Chromatograph (GC) using an RTX-1701 GC column, 30 m long and 0.53 mm ID. Conversions and selectivities are based on normalized areas.

The feed solution was a 4:1 molar ratio of formaldehyde to methyl propionate for all examples. The formaldehyde itself was a 25% solution in ethanol.

TABLE 3

| | | | | | | | | | | % |
|---|---|---|---|---|---|---|---|---|---|---|
| | | | | Feed | | | | | % | Theoretical |
| | | | | Molar | | | | | Conv. | Conv. |
| Ex. | | Feed | Feed | Ratio | N₂ | CT (s) | Temp | TOS | MP to | Based on |
| No. | Catalyst | Solution | cc/hr | F/MP | cc/m | STP | (° C.) | (hr) | MMA | CH₂O |
| 23 | 15% CsOAC on | 4:1 25% | 4 | 4 1 | 48 | 6.15 | 340 | 0.08 | 44.34 | 44.34 |
| | SiO2(grace | CH2O in | | | | | | | | |
| | 55, 12 × 20mesh) | ETOH/MP | | | | | | | | |
| 24 | 15% CsOAC on | 4:1 25% | 4 | 4 1 | 48 | 6.15 | 340 | 0.25 | 46.72 | 46.72 |
| | SiO2(grace | CH2O in | | | | | | | | |
| | 55, 12 × 20mesh) | ETOH/MP | | | | | | | | |
| 25 | 15% CsOAC on | 4:1 25% | 4 | 4 1 | 48 | 6.15 | 340 | 0.5 | 54.10 | 54.10 |
| | SiO2(grace | CH2O in | | | | | | | | |
| | 55, 12 × 20mesh) | ETOH/MP | | | | | | | | |
| 26 | 15% CsOAC on | 4:1 25% | 4 | 4 1 | 48 | 6.15 | 340 | 0.75 | 49.97 | A9.97 |
| | SiO2(grace | CH2O in | | | | | | | | |
| | 55, 12 × 20mesh) | ETOH/MP | | | | | | | | |
| 27 | 15% CsOAC on | 4:1 25% | 4 | 4 1 | 48 | 6.15 | 340 | 1 | 44.22 | 44.22 |
| | SiO2(grace | CH2O in | | | | | | | | |
| | 55, 12 × 20mesh) | ETOH/MP | | | | | | | | |
| 28 | 15% CsOAC on | 4:1 25% | 4 | 4 1 | 48 | 6.15 | 340 | 1.25 | 41.83 | 41.83 |
| | SiO2(grace | CH2O in | | | | | | | | |
| | 55, 12 × 20mesh) | ETOH/MP | | | | | | | | |
| 29 | 15% CsOAC on | 4:1 25% | 4 | 41 | 48 | 6.15 | 340 | 1.5 | 39.36 | 39.36 |
| | SiO2(grace | CH2O in | | | | | | | | |
| | 55, 12 × 20mesh | ETOH/MP | | | | | | | | |
| 30 | 15% CsOAC on | 4:1 25% | 4 | 4 1 | 48 | 6.15 | 340 | 2 | 35.55 | 35.55 |
| | SiO2(grace | CH2O in | | | | | | | | |
| | 55, 12 × 20mesh) | ETOH/MP | | | | | | | | |
| 31 | 15% CsOAC on | 4:1 25% | 4 | 4 1 | 48 | 6.15 | 360 | 0.08 | 70.63 | 70.63 |
| | SiO2(grace | CH2O in | | | | | | | | |
| | 55, 12 × 20mesh) | ETOH/MP | | | | | | | | |
| 32 | 15% CsOAC on | 4:1 25% | 4 | 4 1 | 48 | 6.15 | 360 | 0.25 | 62.38 | 62.38 |
| | SiO2(grace | CH2O in | | | | | | | | |
| | 55, 12 × 20mesh) | ETOH/MP | | | | | | | | |
| 33 | 15% CsOAC on | 4:1 25% | 4 | 4 1 | 48 | 6.15 | 360 | 0.05 | 40.54 | 40.54 |
| | SiO2(grace | CH2O in | | | | | | | | |
| | 55, 12 × 20mesh) | ETOH/MP | | | | | | | | |
| 34 | 15% CsOAC on | 4:1 25% | 4 | 4 1 | 48 | 6.15 | 380 | 0.08 | 42.87 | 42.87 |
| | SiO2(grace | CH2O in | | | | | | | | |
| | 55, 12 × 20mesh) | ETOH/MP | | | | | | | | |
| 35 | 15% CsOAC on | 4:1 25% | 4 | 4 1 | 48 | 6.15 | 380 | 0.25 | 44.10 | 44.10 |
| | SiO2(grace | CH2O in | | | | | | | | |
| | 55, 12 × 20mesh) | ETOH/MP | | | | | | | | |
| 36 | 15% CsOAC on | 4:1 25% | 4 | 4 1 | 48 | 6.15 | 380 | 0.5 | 36.82 | 36.82 |
| | SiO2(grace | CH2O in | | | | | | | | |
| | 55, 12 × 20mesh) | ETOH/MP | | | | | | | | |

What is claimed is:

1. A process for preparing ethylenically unsaturated acids, the process comprising contacting an alkanoic acid of the formula R′—CH₂—COOR, with formaldehyde in the presence of a heterogeneous basic catalyst, and optionally in the presence of an alcohol; wherein R and R′ are each, independently, hydrogen or an alkyl group with 1 to 4 carbon atoms, and wherein the catalyst concentration is from 12% to 20% by weight of the reactants.

2. A process for preparing ethylenically unsaturated esters, the process comprising contacting an ester of an alkanoic acid of the formula R′—CH₂—COOR, with formaldehyde in the presence of a heterogeneous basic catalyst, and optionally in the presence of an alcohol; wherein R and R′ are each, independently, hydrogen or an alkyl group with 1 to 4 carbon atoms, and wherein the catalyst concentration is from 12% to about 20% by weight of the reactants.

3. The process of claim 1 further comprising contacting the alkanoic acid and the formaldehyde in the presence of the basic catalyst and an alcohol.

4. The process of claim 1 further comprising contacting the ester of an alkanoic acid and the formaldehyde in the presence of the basic catalyst and an alcohol.

5. The process of claim 1 or claim 2 wherein the catalyst is selected from the group consisting of a metal silicate, a metal carbonate, a metal oxide, a metal hydroxide, a metal phosphate, a metal aluminate and combinations thereof.

6. The process of claim 1 or claim 2 wherein the catalyst is selected from the group consisting of a Group 1, Group 2 or rare earth silicate, a Group 1, Group 2 or rare earth oxide, a Group 1, Group 2 or rare earth carbonate and combinations thereof.

7. The process of claim 1 or claim 2 wherein the catalyst is selected from the group consisting of barium, cesium, rubidium and magnesium.

8. The process of claim 1 or claim 2 wherein the catalyst is supported on a catalyst support.

9. The process of claim 8 wherein the catalyst support is selected from the group consisting of carbon, alumina, silica, silica-alumina, titania, barium sulfate, compounds thereof, and combinations thereof.

10. The process of claim 1 wherein the alkanoic acid is propionic acid.

11. The process of claim 2 wherein the ester of an alkanoic acid is methyl propionate.

12. The process of claim 1 or claim 2, wherein the alcohol is methanol or ethanol.

13. The process of claim 1 or claim 2, wherein the formaldehyde to alcohol ratio is from 1/4 to 4/1.

14. The process of claim 1 or claim 2 wherein the formaldehyde is contacted in the form of formalin.

15. The process of claim 1 or claim 2 performed in the gas phase.
